(19) United States
(12) Patent Application Publication
(10) Pub. No.: US 2023/0293017 A1
(43) Pub. Date: Sep. 21, 2023

(54) SLEEPING OR RECLINING PIECE OF FURNITURE WITH A SENSOR AND METHOD FOR DETECTING PHYSIOLOGICAL PARAMETERS OF A PERSON

(71) Applicant: DEWERTOKIN GMBH, Jiaxing (CN)

(72) Inventor: Armin HILLE, Bielefeld (DE)

(21) Appl. No.: 18/007,798

(22) PCT Filed: Jun. 2, 2021
(86) PCT No.: PCT/EP2021/064784

§ 371 Date: Dec. 2, 2022

(30) Foreign Application Priority Data
Jun. 4, 2020 (DE) 20 2020 103 225.9

(51) Int. Cl. A61B 5/01; G01J 5/00; G01J 5/02; A61B 5/00

(52) U.S. Cl. CPC A61B 5/01; G01J 5/0025; G01J 5/025; A61B 5/6891; A61B 5/0008; G01J 2005/0077

(57) ABSTRACT

A sleeping or reclining piece of furniture includes a sensor for detecting physiological parameters of a person using the sleeping or reclining piece of furniture. The sensor is at least one thermal imaging camera arranged on the sleeping or reclining piece of furniture. The sleeping or reclining piece of furniture has an analysis unit connected to the at least one thermal imaging camera in order to process image and/or temperature information of the at least one thermal imaging camera.

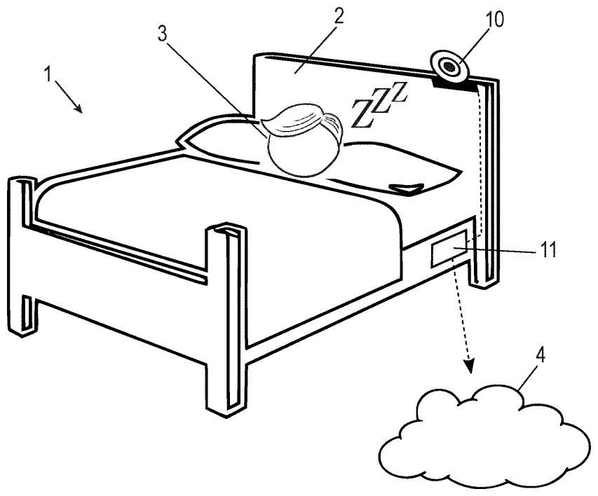

### BACKGROUND AND SUMMARY OF THE INVENTION

Exemplary embodiments of the invention relate to a sleeping or reclining piece of furniture with a sensor and a method for detecting physiological parameters of a person using the sleeping or reclining furniture.

In the clinical field, monitoring devices are known to monitor a patient's health status during sleep in order to intervene, for example, when cardiac function and circulatory parameters are of concern.

Devices for monitoring sleep status based on physiological parameters are now also commercially available for non-clinical purposes. These devices, which are placed on a bedside table, for example, record sounds and/or movement states during sleep with the aid of microphones and/or cameras. The recorded information is used to derive a sleep state, the temporal course of which is recorded. The recorded sleep history can be retrieved and evaluated at a later date. It can provide information about how deep and restful the sleep was.

In addition to systems that use a camera and/or microphone, a sensor-based system is known in which a pressure-sensitive sensor strip is placed over the mattress and in which this sensor strip is connected to a cell phone (smartphone) that records the sensor data. The sensor data is used to derive, among other things, a heart rate and a respiratory rate.

WO 2017/114950 A1 discloses a sleeping or reclining furniture in which a sensor for the detection of vibrations, movement and/or sound is mounted on a section of a supporting element of the sleeping or reclining furniture, which is decoupled from further sections of the supporting element of the furniture. In this way, good detection of the vibrations or sound of the person is achieved even with good suppression of extraneous sound, in particular impact sound. An evaluation of the vibrations or the sound makes it possible, for example, to determine a heart rate, a breathing rate and/or a state of movement of the person using the sleeping or reclining furniture.

In many cases, the physiological parameters mentioned already reflect a state of health of the person using the sleeping or reclining furniture and can provide indications as to whether there is a health risk for the person and whether, if necessary, a warning is issued to a caregiver or a care service. However, it would be advantageous to further expand the possibilities of monitoring a health condition of the person using the sleeping or reclining furniture and also to extend them to symptoms of illness that cannot be detected acoustically.

Accordingly, exemplary embodiments of the present invention are directed to a sleeping and/or reclining furniture of the type mentioned at the beginning, which is capable of determining physiological parameters that cannot be detected via vibrations or sound. It is a further object to specify a method for detecting the physiological parameters.

A sleeping or reclining furniture according to the invention is characterized in that a thermal imaging camera is arranged as a sensor on the sleeping or reclining furniture. A person using the sleeping and/or reclining furniture can thus be detected by the thermal imaging camera and information from the thermal imaging camera can be evaluated to determine physiological parameters of the person, e.g., to determine a change in body temperature without contact or to determine a movement pattern of the person during sleep.

The integration of the thermal imaging camera into the furniture simplifies a wiring of the thermal imaging camera with the sleeping and reclining furniture and especially with components such as an analysis unit located in or on the furniture and leads to a more robust and also tamper-proof system.

A thermal imaging camera is any device that can record and display temperature information in a non-contact, spatially resolved manner. This can be realized by directing heat radiation through suitable optics onto a flat sensor with a plurality of picture elements (pixels). Alternatively, thermal radiation can also be directed through suitable optics in conjunction with deflecting and thereby scanning elements onto a single sensor, e.g., a pyrometric sensor. Advantageously, the at least one thermal imaging camera has a detection angle range of at least 30° and preferably at least 60° to enable detection even in a wide variety of positions in which a user is in the sleeping or reclining furniture.

For example, the thermal imaging camera can be arranged on a headboard of the sleeping or reclining furniture, in particular if the sleeping or reclining furniture is a bed, preferably a stationary bed with feet or lockable castors. If the sleeping or reclining furniture is designed as a nursing bed, advantageously at least two thermal imaging cameras may also be provided, each of which is arranged on a side rail of the nursing bed. In all cases, the at least one thermal imaging camera is further preferably arranged flexibly with regard to its position and/or orientation on the sleeping or reclining furniture so that it can be oriented in the best possible way and by taking into account individual sleeping positions.

The sleeping or reclining furniture further comprises an analysis unit connected to the at least one thermal imaging camera in order to process image and/or temperature information of the at least one thermal imaging camera. Preferably, the analysis unit is designed to evaluate temporal changes in the image and/or temperature information of the at least one thermal imaging camera and is set up to determine a body temperature of the person as a physiological parameter. The analysis unit may further be set up to store the information of the thermal imaging camera and/or the determined physiological parameter at least for a limited period of time. For this purpose, the analysis unit comprises, for example, a memory for storing a time history of the physiological parameters.

The analysis unit can further be designed for connection to a network, preferably an intranet or the Internet, in order to forward the recorded and/or derived information. For this purpose, the analysis unit has a transmission unit for transmitting the physiological parameters to a network and/or to a mobile device. The transmission unit can preferably be set up for wireless transmission of the physiological parameters to the network or to the mobile device, in particular via a WLAN or Bluetooth transmission link. In this case, storage and/or evaluation can be performed alternatively or additionally via network-based services (cloud services). The use of external storage services offers high availability and also long storage duration of the data. The data can easily be made available to various other instances for evaluation or comparison. Such a cloud can be a storage space offered by an external service provider, which is provided in a decentralized and/or distributed manner by servers that are accessible via the Internet. On the other hand, it can also be a so-called personal cloud, in which a storage location is provided locally, e.g., in the form of a NAS (Network Attached Storage) storage device that is accessible on an intranet. Finally, a mass storage device directly connected by cable to the analysis unit would also be understood as a cloud in this sense. Other forms of a cable-based, or more precisely a wired, cloud include USB mass storage sticks or memory cards such as SD cards.

In a further advantageous design of the sleeping or reclining furniture, the analysis unit or a mobile device to which information from the analysis unit is transmitted has a monitoring device for comparing the physiological parameters with predefined limit values. In this way, a warning and alarm function can be implemented, e.g., to call for help in the event of detected deviations of the physiological parameters.

In a further advantageous design, the sleeping or reclining furniture has at least one further sensor, in particular a vibration or sound sensor and/or a camera sensitive in the visible spectral range, wherein this further sensor is also connected to the analysis unit. The analysis unit is then set up to determine at least one further physiological parameter of the person using the sleeping or reclining furniture on the basis of information from the further sensor. Irregularities or illnesses can be detected more reliably in this way because illnesses are usually manifested by various symptoms.

In a further advantageous design, the sleeping or reclining furniture further has an electromotive furniture drive with adjustment drives for adjusting furniture parts and a control device for controlling the adjustment drives, with the analysis unit being coupled to the control device or integrated in the control device. In this way, components of the control device that are already present in the electromotive furniture drive can also be used for the analysis unit, for example a power supply unit, communication devices and/or a housing including connection options. In addition, wiring of the thermal imaging camera is simplified if the existing structure of the electromotive furniture drive is used. Further preferably, the analysis unit can then be integrated into the control device of the electromotive furniture drive. In addition, information sent to the control device of the electromotive furniture drive can be used directly by the latter to control suitable actions of the furniture drive. For example, it may be provided that when certain medical conditions are detected, an adjustment motor of the electromotive furniture drive is activated to change the position of, for example, a back or foot part of the bed, whereby, as a rule, the person in the bed also changes his sleeping position and assumes a soothing position. Further, it can be provided, for example, to temporarily switch on a lighting device coupled to the control device, e.g., a so-called under-bed lighting device, if on the basis of the data sent to the control device it is indicated that the person has just left or left the bed.

A method according to the invention for detecting physiological parameters of a person using such a sleeping or reclining furniture comprises the following steps: A sequence of images of a person located in the sleeping or reclining furniture is captured by the thermal imaging camera. Further, at least one image region is identified in which a temperature can be associated with a body temperature of the person. Then, a temporal change of the temperature and/or position of the identified image area is determined.

Thus, in the method according to the invention, the person is first detected in the thermal image based on plausibility. Objects with a body temperature outside an expected range are not taken into account. The limits can be selected so wide that the method can be carried out even if an exact calibration of the thermal imaging camera is not available. Then an identified image area is observed with regard to changes in the measured temperature and/or position in order to be able to make statements about a state of health.

Although the method does not require an exact temperature calibration, such a calibration can increase the validity of the observations. In an advantageous further development of the method, the method comprises the following additional steps: a display device is coupled to the analysis unit connected to the thermal imaging camera and an image of the thermal imaging camera is displayed on the display device. A body area of the person on the image is then selected, for example by caregivers, and a body temperature is measured by means of a thermometer in this body area of the person. The body area is selected on the image displayed on the display device and the measured body temperature is entered on an input device coupled to the display device. A temperature scale for images of the thermal imaging camera is calibrated based on the selected body area and the input body temperature. A mobile device, for example, can be used as the display device and input device.

This calibration procedure can advantageously be carried out easily and with the aid of a thermometer usually available in the care area, e.g., a non-contact infrared thermometer.

### BRIEF DESCRIPTION OF THE SOLE DRAWING

The invention is explained in more detail below by means of exemplary embodiments with the aid of a FIGURE.

The sole FIGURE shows a schematic representation of a bed with a thermal imaging camera mounted on it.

### DETAILED DESCRIPTION

The sole FIGURE shows a schematic view of a bed 1 as an example of a piece of bedroom furniture.

The bed 1 has a frame with an attached mattress. A thermal imaging camera 10 is arranged on a headboard 2 of the frame. The thermal imaging camera 10 detects a temperature distribution without contact for a certain number of picture elements (pixels) in a detection angle range specified by the optics of the thermal imaging camera 10.

The thermal imaging camera 10 can be integrated in a component of the bed 1 during manufacture of the bed 1, or it can be attached to the furniture during manufacture of the bed 1 or subsequently. The thermal imaging camera 10 is oriented such that a person 3 lying in the bed 1 is positioned with his head within the detection range of the thermal imaging camera 10. In order to allow sufficient flexibility for the detection of the person 3, the thermal imaging camera 10 has a detection angle range of at least 30°, preferably at least 60° and particularly preferably over 100°.

The positioning of the thermal imaging camera 10 on the head section 2 of the bed 1 is shown in the sole FIGURE purely as an example. By taking into account the mounting location of the thermal imaging camera 10, the detection angle range is selected so that in any usual positioning of the person 3, the head is detected by the thermal imaging camera.

In alternative designs, the thermal imaging camera 10 can also be mounted at other positions, in particular on a side rail of the bed in the case of healthcare beds. It is also conceivable to arrange more than one thermal imaging camera 10 on the bed 1. In particular, if a picture is taken from the side, it is useful to use at least two thermal imaging cameras 10, each oriented from one side toward the center of the bed 1.

It is also conceivable that the at least one thermal imaging camera 10 is mounted on a piece of furniture that forms a unit belonging together with the sleeping or reclining furniture, for example a side table or nightstand. The side table or nightstand are to be understood in the sense as part of the sleeping or reclining furniture.

In addition, a thermal imaging camera that is not directly mounted on the sleeping or reclining furniture or a piece of furniture belonging to it, but is assigned to it, can be provided, the thermal images of which are evaluated as a supplement. For example, a thermal imaging camera mounted on a wall opposite the bed but aligned with the bed can be used.

In all of the above cases, it may be provided that the thermal imaging camera 10 is arranged in an adjustable manner with respect to its position and/or orientation. For this purpose, it can be fixed, for example, with the aid of clamps or latching arrangements in order to vary its position. It is also conceivable to provide a pivoting arrangement in one or more spatial directions in order to be able to change an orientation of the camera. Another possibility is to arrange the camera on a flexible holder, for example a gooseneck holder, which allows variation of both the positioning and the orientation.

In the exemplary embodiment shown, the thermal imaging camera 10 is connected to an analysis unit 11 also arranged on the bed 1. In an advantageous alignment of the thermal imaging camera 10, the head of the person 3 in particular is positioned as centrally as possible in the thermal image. To achieve this, the analysis unit 11 can support the nursing staff in various ways. For example, it can be provided that the analysis unit 11 transmits the thermal image for display to a display device, with the aid of which the thermal imaging camera 10 can be aligned. The display device may be, for example, a mobile device (e.g., smartphone or tablet computer) of the caregiver.

Alternatively, or additionally, an automatic image evaluation can be performed in the analysis unit 11, which e.g., determines a center of gravity of a temperature distribution indicating a body temperature and indicates via simple display elements such as light emitting diodes (LED) whether the center of gravity is approximately in the center of the image. For example, a green LED may indicate a correct orientation and optionally a plurality of, for example, red directional arrows may indicate in which direction the thermal imaging camera 10 must be pivoted to arrive at the correct orientation.

The analysis unit 11 evaluates the image and temperature data supplied by the thermal imaging camera 10, stores them at least for a limited period of time and derives information about physiological parameters of the person 3. The analysis unit 11 is preferably connected to a network 4, preferably an intranet or the Internet, in order to forward the captured and/or the derived information.

The analysis unit 11 can, for example, be connected to external storage via the network 4. On the one hand, this can be a cloud, e.g., a storage location offered by an external service provider, which is provided in a decentralized and/or distributed manner by servers that can be accessed via the Internet. On the other hand, it can also be a so-called personal cloud, in which a storage location is provided locally, e.g., in the form of a NAS (Network Attached Storage) storage device that is accessible on an intranet. Finally, a mass storage device directly connected by cable to the analysis unit would also be understood as a cloud in this sense. Other forms of a cable-based, or more specifically, a wired cloud include USB mass storage sticks or memory cards such as SD cards. These cloud-forming storage elements can be provided in various locations and components, including, for example, in a PC (personal computer) or a smartphone as a mobile device.

The analysis unit can also have a monitoring device for comparing the physiological parameters with predefined limit values, so that in a case where a health risk to the person is detected, the person or another person can be warned. For this purpose, the analysis unit preferably has a signal device for outputting a warning signal.

Alternatively, it may be provided to output a warning message via the network 4 so that it is output on a paired device, e.g., a mobile device of a caregiver or a care service. Instead of the connection to the network 4, there may also be a transmission unit in the analysis unit 11 that transmits a warning message directly to a mobile device or other external device. The transmission unit is then preferably set up for wireless transmission to the mobile device or other external device, in particular via a WLAN or Bluetooth transmission link. A wired coupling to external units is also conceivable, for example if the external unit is a staff call system in a nursing home. The warning message issued via the network 4 can have a multi-stage design and include, for example, an alarm pre-stage that is already triggered when the person 3 approaches an image edge of the thermal image. This can be seen as a request to check the position of the person 3 in the bed 1 and also to check the correct orientation of the thermal imaging camera 10. A next alarm stage can then be triggered, for example, when the person 3 is actually no longer identified in the image area of the thermal imaging camera 10.

It may also be provided to transmit the physiological parameters, i.e., at least the determined body temperature itself, to the mobile device or another external unit. A comparison of the physiological parameters with predefined limit values can then also be carried out externally by the analysis unit 11.

Alternatively, the monitoring device itself can be designed externally to the analysis unit and be connected to the analysis unit. Such an external monitoring device can be formed in a mobile device, for example. The functionality required for this can be provided via a corresponding program (“app”). The external monitoring device can also be part of an alarm control center, for example in a care facility.

Preferably, information from the thermal imaging camera 10 is not evaluated in absolute terms, but with regard to observed changes. In this way, important information about the physiological condition of the person 3 can be obtained even without an exact calibration of the thermal imaging camera 10.

For the evaluation, plausibility classifications are first used to detect whether a detected temperature pattern can be assigned to a person at all. For this purpose, a spatial and/or temporal change behavior of the detected data can be used. For example, an identified periodically increasing and decreasing heat source may indicate a cigarette and a very suddenly occurring and disappearing heat source may indicate a lighter or a hot beverage cup picked up briefly from the bedside table, for example, and put down again after drinking. In contrast, a heat source with a briefly constant temperature that changes only slightly over a longer period of time can be assigned to a person.

Although temperature change in particular is preferred and not absolute measured temperatures, absolute measured values can be used in extreme cases to identify heat sources. In this regard, in particular, measured data suggesting temperatures significantly above body temperature, even if the thermal imaging camera is not precisely calibrated, and physiological and therefore more likely to be associated with other heat sources such as the aforementioned cigarettes, lighters (or even a fire) and/or beverage cups.

If a person, for example the person 3 shown in the sole FIGURE, is identified as a probable heat source, this already provides information as to whether a person is in the bed at all or has just left it. This information is also valuable in itself and can be passed on as warning information by the aforementioned monitoring device if necessary.

A temperature can be recorded from a detected person in order to observe and meaningfully evaluate a change in temperature.

A gradual increase in temperature, either in spots or over a wide area, indicates the development of an illness with fever symptoms. Typical temperature changes that people show due to different sleep phases can be taken into account.

With the detection of the person 3 by the thermal imaging camera 10, a movement pattern of the person 3 can also be recorded, which is analyzed and, optionally, additionally allows conclusions to be drawn about a physiological state of the person 3. An identification of the sleep phases can be made, for example, on the basis of the previously mentioned movement state of the person 3. Movement states that deviate from typical sleep patterns also allow conclusions to be drawn about illnesses.

Even though preferably the temperature information output by the thermal imaging camera 10 is not analyzed in absolute terms but with respect to its change over time, a calibration of the thermal imaging camera 10 may be performed and may be helpful, in particular to be able to reliably identify heat sources and distinguish between them.

For this purpose, it can be provided, for example, that a calibration measurement is carried out with the aid of a thermometer, e.g., by nursing staff, on a body part that is recognizable and easily identifiable on the thermal image, e.g., on the forehead of the person 3. For this purpose, the thermal image is transmitted from the analysis unit 11, e.g., to the mobile device of the nursing staff. This can be carried out, for example, by coupling with the mobile device via the network 4 or directly via WLAN or Bluetooth. A contactless method is preferably used for temperature measurement, for example by means of an infrared thermometer.

The mobile device can compare the measured value with the specifications of the (current) camera calibration. In case of deviation, the measuring point or the area corresponding to the measuring point can be selected on the display of the thermal image and the measured value of the thermometer can be entered. This value is taken over for calibration and transferred accordingly to all other areas of the thermal image.

Alternatively, or additionally, it may be provided that a camera sensitive in the visible spectral range of light can be provided in addition to the thermal imaging camera. This camera can be used to record the skin color of the person, which allows conclusions to be drawn about the presumed emission factor of the skin surface. Furthermore, environmental values such as a room temperature and/or a degree of humidity can be measured by corresponding sensors and correction values for the temperature information output by the thermal imaging camera 10 can be derived from this using known algorithms.

It may further be provided that the bed 1 has an electromotive furniture drive with at least one adjustment drive for adjusting one or more movable furniture parts. Such an electromotive furniture drive, which is also frequently found in nursing beds, for example, generally has a control device which receives control instructions from one or more manual control units and controls the adjustment drives. In such a design, the analysis unit 11 can preferably be coupled to the control device or integrated into the control device.

In this way, already existing components of the control device of the electromotive furniture drive can also be used for the analysis unit, for example a power supply unit, communication devices and/or a housing including the connection options. In addition, wiring of the thermal imaging camera 10 is simplified if the existing structure of the electromotive furniture drive is used.

In a further development of the arrangement shown in the sole FIGURE, further sensors can be arranged on the bed 1 or assigned to it in addition to the thermal imaging camera 10 and coupled to the analysis unit 11. For example, vibration or sound sensors for recording body or airborne sound can be connected to the analysis unit 11. Such a sound sensor may be formed, for example, by a condenser microphone. Piezo elements may be provided as vibration sensors. The recording of vibrations and/or airborne sound allows pulse and respiration frequencies to be determined.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the FIGURES enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### LIST OF REFERENCE SIGNS

1 Bed
2 Headboard
3 Person
4 Network
10 Thermal imaging camera
11 Analysis unit

1-17. (canceled)

18. A sleeping or reclining furniture, comprising:
a sensor configured to detect physiological parameters of a person using the sleeping or reclining furniture, wherein the sensor is at least one thermal imaging camera arranged on the sleeping or reclining furniture; and
an analysis unit connected to the at least one thermal imaging camera to process image or temperature information of the at least one thermal imaging camera.

19. The sleeping or reclining furniture of claim 18, wherein the analysis unit is configured to evaluate temporal changes in the image or temperature information of the at least one thermal imaging camera.

20. The sleeping or reclining furniture of claim 19, wherein the analysis unit is configured to determine a body temperature of the person as a physiological parameter.

21. The sleeping or reclining furniture of claim 20, further comprising:
at least one further sensor connected to the analysis unit, wherein the at least one further sensor is a vibration sensor, sound sensor, or a camera sensitive in a visible spectral range, wherein the analysis unit is configured to determine at least one further physiological parameter of the person based on information from the further sensor.

22. The sleeping or reclining furniture of claim 21, wherein the analysis unit comprises a memory configured to store a time course of the physiological parameter and the further physiological parameter.

23. The sleeping or reclining furniture of claim 20, wherein the analysis unit comprises a monitoring device configured to compare the physiological parameter with a predetermined limit value.

24. The sleeping or reclining furniture of claim 20, wherein the analysis unit comprises a transmission unit configured to transmit the physiological parameter to a network or to a mobile device.

25. The sleeping or reclining furniture of claim 24, wherein the transmission unit is configured to wirelessly transmit the physiological parameter to the network or to the mobile device via a WLAN or Bluetooth transmission link.

26. The sleeping or reclining furniture of claim 24, wherein the mobile device includes monitoring means for comparing the physiological parameters to predetermined limits.

27. The sleeping or reclining furniture of claim 18, wherein the at least one thermal imaging camera has a detection angle range of at least 60°.

28. The sleeping or reclining furniture of claim 18, wherein the sleeping or reclining furniture is a bed, and wherein the at least one thermal imaging camera is arranged on a headboard of the bed.

29. The sleeping or reclining furniture of claim 18, wherein the sleeping or reclining furniture is a nursing bed, wherein the at least one thermal imaging camera comprises at least two thermal imaging cameras, each of which is arranged on a side rail of the nursing bed.

30. The sleeping or reclining furniture of claim 18, wherein the at least one thermal imaging camera is flexibly arranged with respect to its position or orientation on the sleeping or reclining furniture.

31. The sleeping or reclining furniture of claim 18, further comprising:
an electromotive furniture drive with adjustment drives configured to adjust furniture parts and a control device configured to control the adjustment drives, wherein the analysis unit is coupled to the control device or is integrated into the control device.

32. A method for detecting physiological parameters of a person in a sleeping or reclining furniture, the method comprising:
capturing a sequence of images from a thermal imaging camera of the person located in the sleeping or reclining furniture;
identifying image areas of the sequence of images in which a temperature can be associated with a body temperature of the person; and
detecting a change in the temperature or a position of the identified image area over time.

33. The method of claim 32, further comprising:
coupling a display device with an analysis unit connected to the thermal imaging camera;
displaying an image of the thermal imaging camera on the display device;
selecting an area of the person's body in the image and detecting a body temperature of the person in that body area using a thermometer;
selecting the body region on the image displayed on the display device and inputting the body temperature on an input device coupled to the display device; and
calibrating a temperature scale for images of the thermal imaging camera based on the selected body area and the entered temperature.

34. The method of claim 33, wherein the display device and the input device are part of a mobile device.
